(19) United States
(12) Patent Application Publication
(10) Pub. No.: US 2012/0259346 A1
(43) Pub. Date: Oct. 11, 2012

(54) THERMAL SUTURE CUTTING DEVICE

(75) Inventors: James Hansen, Coon Rapids, MN (US); Kevin Edmunds, Ham Lake, MN (US); Joel Groff, Montrose, MN (US); Michael Pikus, Golden Valley, MN (US)

(73) Assignee: BOSTON SCIENTIFIC SCIMED, INC., Maple Grove, MN (US)

(21) Appl. No.: 13/440,821

(22) Filed: Apr. 5, 2012

Related U.S. Application Data
(60) Provisional application No. 61/473,412, filed on Apr. 8, 2011.

(51) Int. Cl. A61B 17/04

(52) U.S. Cl. 606/138; 606/232

(57) ABSTRACT

The disclosure pertains to a device for cutting a suture in a fluid environment which isolates a portion of the suture to be cut within a substantially sealed environment and cuts the suture thermally by using an electrically heated element within the chamber. In another embodiment, the disclosure relates to a vascular sealing system including the suture cutting device and a method of use thereof.

### BACKGROUND

In many medical procedures, such as, for example, balloon angioplasty and the like, an opening can be created in a blood vessel or arteriotomy to allow for the insertion of various medical devices which can be navigated through the blood vessel to the site to be treated. For example, a guidewire may first be inserted through a tissue tract created between the skin, or the epidermis, of the patient down through the subcutaneous tissue and into the opening formed in the blood vessel. The guidewire is then navigated through the blood vessel to the site of the occlusion or other treatment site. Once the guidewire is in place, an introducer sheath can be inserted over the guide wire to form a wider, more easily accessible, tract between the epidermis and the opening into the blood vessel. The appropriate medical device can then be introduced over the guidewire through the introducer sheath and then up the blood vessel to the site of the occlusion or other treatment site.

Once the procedure is completed, the medical devices or other equipment introduced into the vessel can be retracted through the blood vessel, out the opening in the blood vessel wall, and out through the tissue tract to be removed from the body. The physician or other medical technician is presented with the challenge of trying to close the opening in the blood vessel and/or the tissue tract formed in the epidermis and subcutaneous tissue. A number of different device structures, assemblies, and methods are known for closing the opening in the blood vessel and/or tissue tract, each having certain advantages and disadvantages. However, there is an ongoing need to provide new and improved device structures, assemblies, and/or methods for closing and/or sealing the opening in the blood vessel and/or tissue tract.

### SUMMARY

This disclosure pertains to a device for cutting a suture in a subcutaneous fluid environment which isolates a portion of the suture to be cut within a substantially sealed environment, thereby excluding the surrounding fluid environment, and cuts the suture thermally by using an electrically heated element within the chamber. In a first embodiment, this disclosure pertains to a thermal suture cutting device for use with vascular sealing devices comprising a chamber having a first compression bead defining an first opening for the suture, the opening sized to slidingly receive a suture to be cut; a second compression bead defining an second opening sized and adapted to slidingly and sealingly receive the suture to be cut, an electrically activated heating element within the chamber, said electrically activated heating element being capable of generating a temperature within the chamber greater than the melting temperature of the suture to be cut; and electrical leads at least partially within the chamber capable of supplying sufficient current to the electrically activated heating element to allow the electrically activated heating element to generate a temperature within the chamber greater than the melting temperature of the suture to be cut; a push rod having a proximal end and a distal end, said distal end being attached to the first compression bead component, and one or more lumens extending at least partially between said proximal and distal end; and a source of electrical current sufficient to allow the electrically activated heating element to generate the temperature within the chamber greater than the melting temperature of the suture to be cut. The source of electrical current is connected to the electrical leads and includes a switch capable of controlling the flow of electrical current. The electrical current source is associated with a handle at the proximal end of the push rod. The first compression bead and second compression bead cooperate with the suture to be cut to form a substantially fluid tight chamber within which the suture may be cut, said chamber being capable of excluding a surrounding fluid environment.

In another embodiment, this disclosure relates to a system for sealing a vascular puncture comprising an anchor deployable adjacent to a vascular puncture; a suture attached to the anchor deployable adjacent to the vascular puncture; a hemostatic material deployed about the suture; and the thermal suture cutting device described herein.

In yet another embodiment, this disclosure relates to a method of cutting a suture in a fluid environment, such as blood or other body fluid, comprising passing a suture through the thermal suture cutting device described above; positioning the suture and the thermal suture cutting device in the fluid environment; and passing a current through the electrically activated heating element of the device sufficient to allow the electrically activated heating element to generate a temperature within the chamber greater than the melting temperature of the suture to be cut.

In these embodiments, the thermal suture cutting device isolates a portion of the suture to be cut from a surrounding fluid environment and thermally cuts the suture with a minimal consumption of energy.

### BRIEF DESCRIPTION OF DRAWINGS

FIG. 1 illustrates components of an exemplary thermal suture cutting device.

FIG. 2A illustrates an embodiment of a portion of an exemplary thermal suture cutting device.

FIG. 2B illustrates an embodiment of a portion of another exemplary thermal suture cutting device.

FIGS. 3A-3C illustrate configurations of an electrically activated heating element.

FIG. 4 illustrates elements of a system for sealing a vascular puncture.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless cleared stated to the contrary.

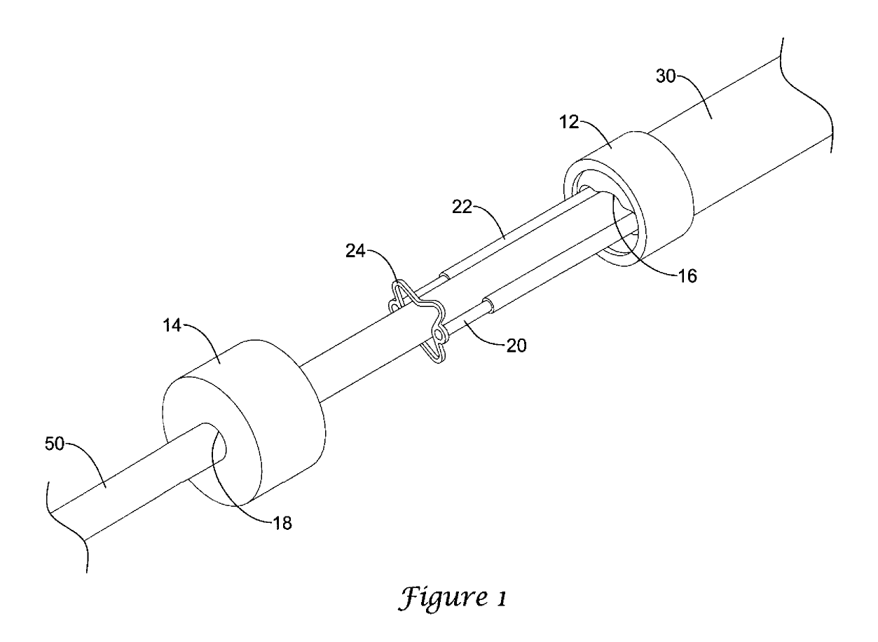

FIG. 1, illustrates a thermal suture cutting device comprising a first compression bead component 12, a second compression bead component 14, an electrically activated heating element 24; and a push rod 30. Compression bead components 12, 14 form the distal end of suture cutting device and may be used to position and compress a plug of hemostatic material (not shown) adjacent to a vessel puncture. The first compression bead component 12 defines an opening 16 sized and adapted to sliding receive a suture 50 as well as passages for wires 20 and their insulation 22. The first compression bead component 12 may be fixedly attached to a push rod 30 which also includes at least one lumen (not shown) for the suture 50, and optionally additional lumens for wires 20, and insulation 22. In some embodiments, the push rod 30 and the first compression bead component 12 may be fabricated from a single piece. In other embodiments, the push rod 30 may be attached to the first compression bead component 12 by a coupling (not shown) which allows a minor amount of deflection therebetween. In yet other embodiments, insulation 22 may be omitted if the push rod 30 is formed from an insulating material. In some such embodiments, the wires 20 may be embedded directly in the material of the push rod 30.

Second compression bead component 14 also defines an opening 18 sized and adapted to sliding receive the suture 50. In some embodiments, opening 18 can also be sized and adapted to sealingly receive suture 50. The sealing capability, if present, associated with opening 18 may be provided by sizing the opening 18 to lightly compress the suture 50 or may be provided by an elastomeric seal (not shown). In some embodiments the sealing capability may be provided by contact with a cinch button (not shown) associated with the suture 50. In other embodiments the sealing capability may be provided by a pressure differential between the interior of the device and a fluid of a fluid environment surrounding the first and second compression bead components 12, 14. In yet other embodiments the sealing capability may result from a selection of material for one or both of the second compression bead components 12, 14 and the suture such that surface tension of the fluid of the fluid environment substantially prevents the fluid of the fluid environment from entering the opening 18. Other sealing means may also be used.

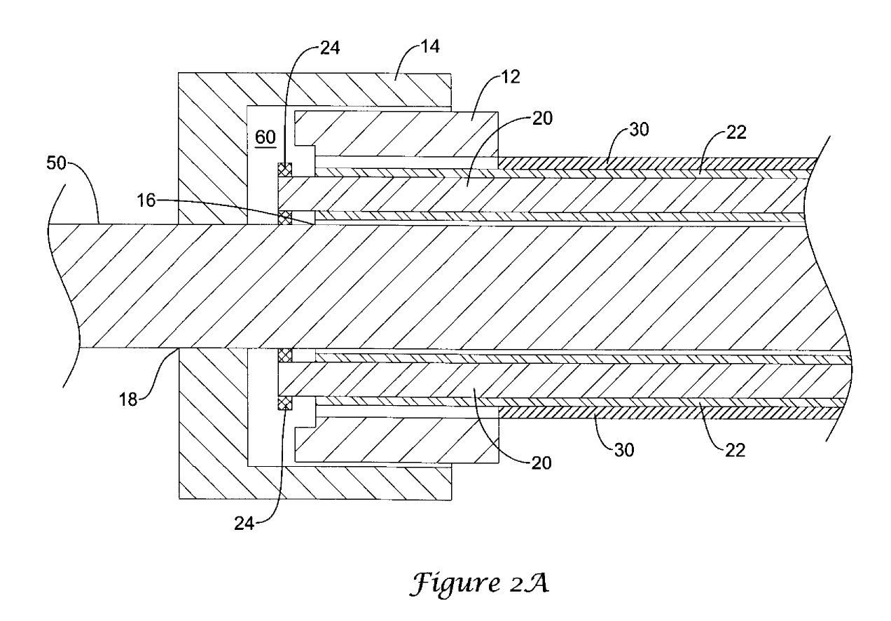

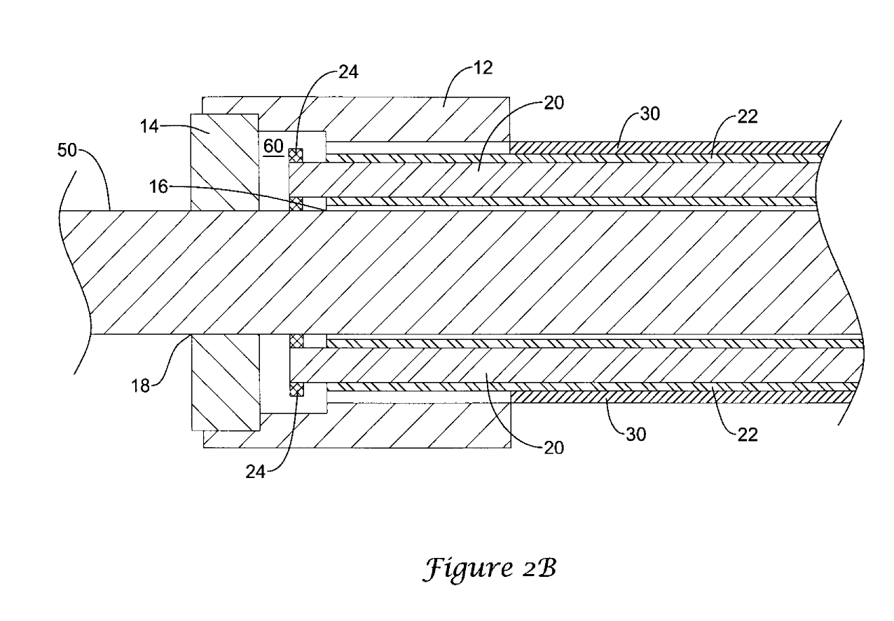

As illustrated in FIGS. 2A and 2B, first compression bead component 12 and the second compression bead component 14, in cooperation with suture 50 and any associated sealing elements (not shown), serve to define a chamber 60 within the space between the first and second compression bead components 12, 14, which remains substantially free of the fluid of the external fluid environment at least until the suture 50 has been cut by the electrically activated heating element 24.

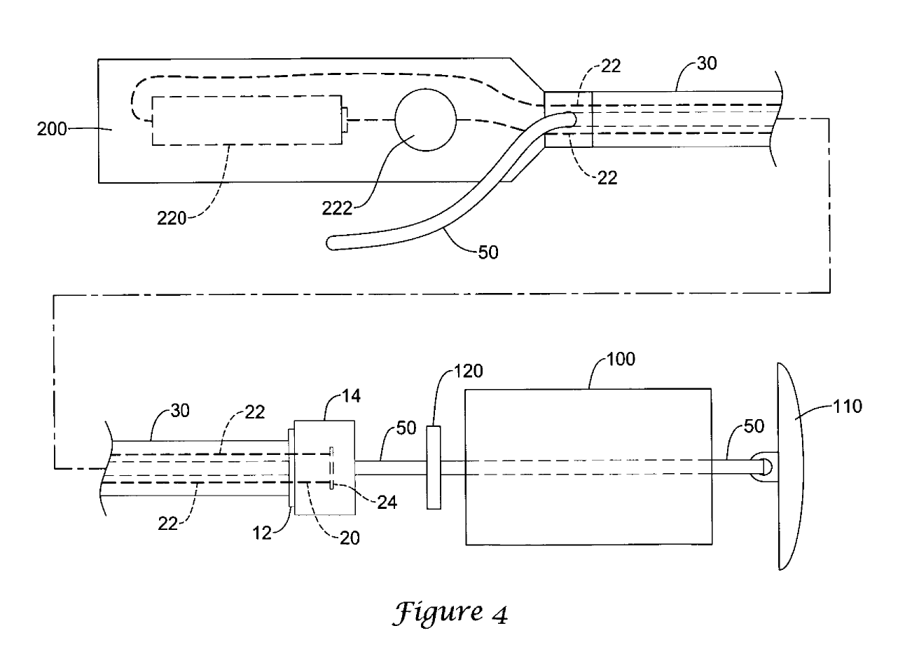

Electrically activated heating element 24 can be positioned within chamber 60 and proximate suture 50. Electrically activated heating element 24, capable of generating a temperature within the chamber 60 greater than the melting temperature of the suture 50 to be cut, is connected to electrical leads, or wires 20, which are connected to a source of electrical current sufficient to allow the electrically activated heating element to generate the temperature within the chamber 60 of greater than the melting temperature of the suture to be cut. The source of electrical current may include a switch 222 (FIG. 4) which is capable of connecting and/or disconnecting the source to the wires 20 and thence to the electrically activated heating element 24 such that when the switch is closed, a current flows through the electrically activated heating element 24, heating at least a portion of the interior of the chamber 60 to a temperature greater than the melting temperature of the suture 50 to be cut, whereupon the suture 50 melts. In some embodiments melting of the suture 50 not only cuts the suture 50, but may also result in the formation of a generally ball-shaped tip on the proximal end of the distal portion of the cut suture 50 which may serve as a mechanical stop to prevent the cinch button 120 (FIG. 4), to be discussed further herein, from being displaced when an associated hemostatic plug 100 (FIG. 4) expands. In those embodiments in which a ball-shaped tip is to be formed on the proximal end of the distal portion of the cut suture 50, it may be desirable to employ a somewhat larger opening 18 to allow the ball-shaped tip to pass through.

It will be understood that the terms “cut” and “cutting” as used in this disclosure broadly include related terms such as “melting”, “breaking”, “brittle fracture”, and the like which indicate that the suture 50 is severed thereby. Similarly, the term “suture” should be broadly interpreted to include thread, string, monofilament materials, twisted multifilament materials, braided materials, and the like.

The first and second compression bead components 12, 14 may assume a number of geometries in addition to the non-limiting examples provided. For example, the second compression bead component 14 may take the form of an open cup, as illustrated in FIG. 2A, which mates with and partially surrounds first compression bead component 12 and the electrically activated heating element 24. The first and second compression bead components 12, 14 may form a frictional interference fit, may be bonded together by conventional means, or may be joined by additional joining and/or sealing members (not shown).

In the embodiment illustrated in FIG. 2B, the arrangement of FIG. 2A is generally inverted such that the first compression bead component 12 is a distally facing cup which receives a second compression bead component 14. As in FIG. 2A, first and second compression bead components 12, 14 may form a frictional interference fit, may be bonded together by conventional means, or may be joined by additional joining and/or sealing members (not shown). In some such embodiments the second compression bead may be formed of a biodegradable material and may provide the function of and/or replace cinch button 120.

The first and second compression bead components 12, 14 may desirably be formed from a material or materials which are not adversely affected by exposure to temperatures produced within chamber 60 by the electrically activated heating element 24. For example, first and second compression bead components 12, 14 may be formed from biocompatible metals and/or ceramics. The materials may be cast or machined to their final shapes. In those embodiments in which the second compression bead component 14 also serves as a cinch button 120 which remains after the suture is cut, it may be desirable for the fabrication material to be bioerodible or biodegradable as mentioned above.

Push rod 30 may be formed integrally with the first compression bead component 12 or may be formed separately and joined to the first compression bead component 12 in a later step. The attachment of the push rod 30 to the first compression bead component 12 may be permanent or temporary. Temporary attachment may be desirable in embodiments in which the push rod 30 and an associated handle 200 (FIG. 4), to be discussed herein, are to be reused. Push rod 30 may be rigid or it may be flexible to allow the thermal suture cutting device to better align with other devices with which it may be used. Push rod 30 may be made from the same material as the first compression bead component 12 or it may be made from a different material. For example, the first compression bead component 12 may be made from a metal or ceramic to better resist damage from the heat produced within chamber 60, while the push rod 30 may be made from a polymer for lighter weight and/or to impart somewhat greater flexibility.

Push rod 30 may have one or more partial or complete lumens 32. The lumens 32 may accommodate the suture 50, electrical leads, wires 22, and optionally other devices. Any of the lumens 32 may extend the entire length of the push rod 30 or may terminate along the shaft of the push rod 30. For example, lumen 32 may terminate near the proximal end of push rod 30 to allow tension to be applied to the suture 50 prior to cutting and to allow the proximal portion of suture 50 to be removed following cutting to confirm that the cut has been successful with a well formed end. Alternatively, lumen 32 may terminate near first compression bead component 12 to allow the suture 50 to lie alongside the exterior of push rod 30 which may allow for the use of a smaller and/or more flexible push rod 30.

Similarly, electrical leads or wires 20 may be routed either within optional lumens 32, with suture 50 in a single lumen 32, or externally along push rod 30. Electrical leads or wires 20 may optionally be covered by insulation 22 and/or may be embedded in and/or insulated by the material of push rod 30.

One or more electrically activated heating element(s) 24 can be located within chamber 60 and proximate suture 50. Within the chamber 60, one or more electrically activated heating element(s) 24 can be isolated from the thermal mass of components of the fluid environment outside of the thermal suture cutting device which isolation tends to minimize the energy which would otherwise be required to cut the suture 50. In addition, isolation of the electrically activated heating element 24 from the fluid of the fluid environment outside of chamber 60 reduces the need for protective coatings or insulation on the electrically activated heating element 24 itself which further reduces the energy which would otherwise be required to cut the suture 50.

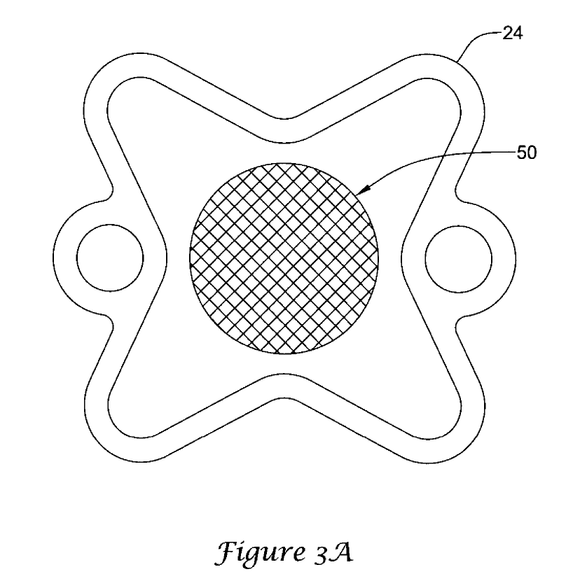

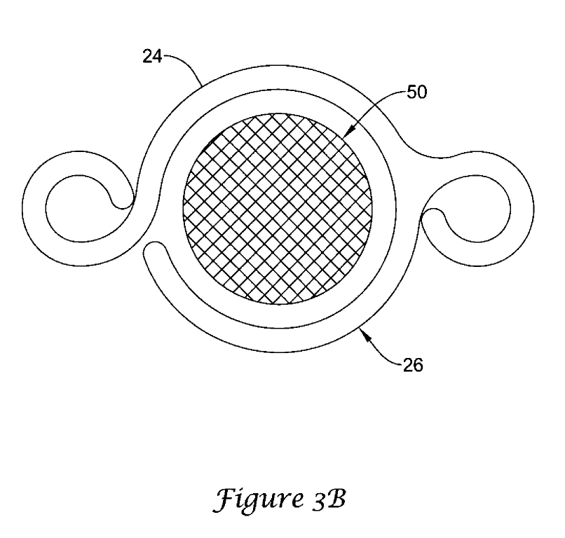

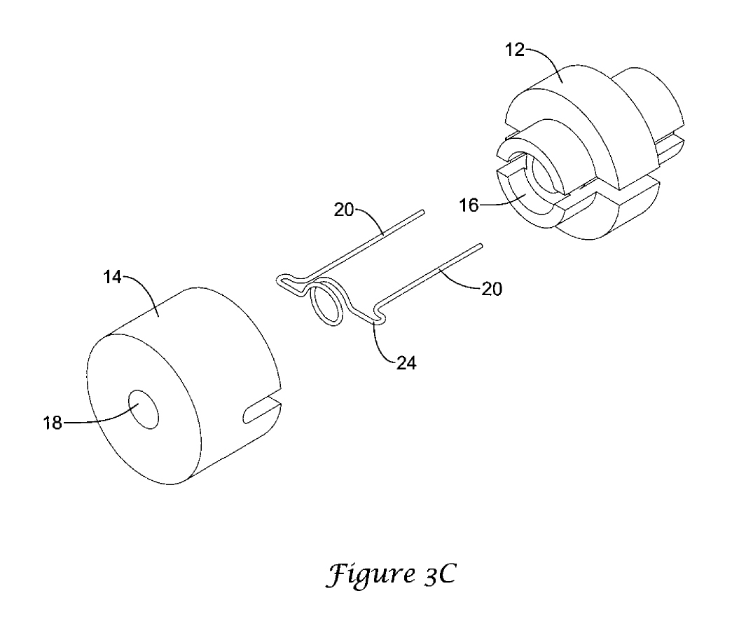

Electrically activated heating element 24 may be fabricated from any of the high resistance materials commonly employed in electrically activated heating elements such as platinum, nichrome, nitinol, tungsten, thick film resistor or thermistor pastes, and the like. The electrically activated heating element(s) 24 may partially or completely surround the suture 50 to be cut although contact is not necessary and may not be desirable. An electrically activated heating element 24 may take any of a number of shapes such as those of the non-limiting examples of FIGS. 3A, 3B, and 3C. It will be appreciated that additional mechanical features within chamber 60 may position or stress the suture to facilitate cutting by one or more electrically activated heating elements 24. In some embodiments, such as that of FIG. 3B, the electrically activated heating element 24 may include mechanical positioning features such as arm 26. Other electrically activated heating elements 24 may take the form of coils, such as illustrated in FIG. 3C, sleeves, parallel bars, and the like. FIG. 3C also illustrates an alternate form of mounting the electrically activated heating element 24 in which bent portions of the heating element 24 engage recesses in the first compression bead component 12. In some embodiments, thermomechanical components of the electrically activated heating element 24 may further serve to cut, melt, or otherwise disrupt the suture 50.

The devices of the present disclosure can have associated therewith a source (not shown) of electrical current capable of supplying sufficient current to the electrically activated heating element 24 to generate a temperature within chamber 60 greater than the melting temperature of the suture 50 to be cut. In alternate embodiments in which the thermally mediated cutting of suture 50 is supplemented by other means, a lesser temperature may suffice. For example, it may suffice to soften the suture 50 when the softened suture 50 is in contact with a displacing sharp edge.

The source 220 of electrical current may include a battery, as illustrated in FIG. 4, or an external power supply. In some case, the source 220 may include circuitry to enhance the current which the device is capable of delivering in a short period of time. In addition, the source 220 of electrical current may include a switch 222 or other means of directly or indirectly controlling the flow of current. In some embodiments, a momentary contact switch 222 will allow manual activation of the electrically activated heating element 24. In other embodiments, the momentary contact switch 222 may activate circuitry which allows current to flow for a specified length of time. In yet other embodiments, switch 222 may be activated by axial pressure applied to one or both of push rod 30 and first compression bead component 12 to ensure that the thermal suture cutting device is properly engaged with a proximal surface of a device to be secured by suture 50.

In some embodiments, the source 220 of current and/or switch 222 or other means of directly or indirectly controlling the flow of current may be housed in a handle 200 for convenient manipulation of the thermal suture cutting device. The handle 200 may be connected directly or indirectly to push rod 30. FIG. 4 illustrates an exemplary system for sealing a vascular puncture which includes handle 200 as well as additional elements of a conventional hemostatic plug 100 and anchor 110 system which may be secured in place by a suture 50. In FIG. 4, elements 100, 110, and 120 are depicted as spread somewhat apart as they might be disposed prior to being urged toward each under the influence of thermal suture cutting device prior to cutting suture 50 proximal of cinch button 120.

Anchor 110 may be positioned in a vessel having a puncture to be sealed such that the suture 50 extends through a plug of hemostatic material 100 and a cinch button 120 or other means of securing the combination of anchor 110 and hemostatic material 100 in their respective positions relative to the vessel wall. In prior art systems, the anchor 110 and hemostatic material 100 have been secured by tying a knot in the suture 50 proximal of the hemostatic material 100 with or without an element analogous to cinch button 120. In systems including a cinch button 120, the knot may be positioned proximal of cinch button 120) which serves to distribute forces generated as the hemostatic material 100 swells in response to contact with blood or other body fluids escaping from the vessel.

The location of a knot or other securing means within a narrow tissue tract which may be filled with fluid tends to make tying and positioning the knot, as well as cutting the suture 50 adjacent to the knot or cinch button 120 difficult. Further, slippage of the knot or tearing of the hemostatic material 100 may lead to undesirable failure of the vascular sealing system.

In use, the system may be advanced within a sheath or simply advanced within a tissue tract adjacent to the punctured vessel. Anchor 110 may be positioned within the vessel and tension applied to the suture 50 to seat the anchor 110 against the vessel wall. In some embodiments, anchor 110 may instead be present in another form such as a hook or hooks or even a simple stitch formed by the suture 50. Hemostatic material, often in the form of a gelatin sponge or pledget, may then be advanced along the suture 50 to a position adjacent to the puncture in the vessel wall. Various means (not shown) may be used to ensure that the hemostatic material 100 is properly positioned relative to anchor 110 and/or the vessel wall. A cinch button 120 may be advanced along the suture 50 and positioned adjacent to hemostatic material 100 where it serves to distribute compressive forces applied by the thermal suture cutting device and/or by the hemostatic material 100 as it expands upon contact with blood or other body fluid. In some embodiments, second compression bead component 14 may provide the function of a cinch button. In such embodiments, the second compression bead component 14 may be formed of a biodegradable or bioerodible material.

In either event, the thermal suture cutting device is advanced along the suture 50 to position and/or slightly compress hemostatic material 100. This may be accomplished by advancing push rod 30 and/or handle 200. It may be desirable to maintain tension on the suture 50 throughout the positioning and cutting processes. This may be accomplished manually by grasping the suture 50 after it exits thermal suture cutting device or additional apparatus (not shown) may apply tension to the suture 50.

When it has been determined that the hemostatic material 100 and the thermal suture cutting device are properly positioned, current may be passed through the electrically activated heating element 24 thereby heating a portion of suture 50 within chamber 60 as described above and cutting suture 50. In some embodiments, cutting the suture may form a ball-shaped tip on the proximal end of the distal portion of the cut suture 50 which may serve as a mechanical stop to prevent the cinch button 120, or second compression bead component 14, from being displaced. The formation of a ball-shaped tip on the suture 50 may greatly increase the force required to displace the cinch button 120 from the suture. Following cutting of suture 50, the thermal suture cutting device and the proximal portion of suture 50 may be removed.

Although the illustrative examples described above relate to cutting a suture which is a component of a vascular sealing system is also contemplated that devices of this disclosure will be useful in cutting sutures, ribbons, or other similar materials submerged in other fluid environments.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A thermal suture cutting device for use with vascular sealing devices comprising:
a chamber further comprising:
a first compression bead component defining a first opening therethrough, said first opening being sized and adapted to slidingly receive a suture to be cut;
a second compression bead component defining a second opening therethrough, said second opening being sized and adapted to slidingly and sealingly receive the suture to be cut;
an electrically activated heating element within the chamber, said electrically activated heating element being capable of generating a temperature within the chamber greater than the melting temperature of the suture to be cut; and
electrical leads at least partially within the chamber capable of supplying sufficient current to the electrically activated heating element to allow the electrically activated heating element to generate a temperature within the chamber greater than the melting temperature of the suture to be cut;
a push rod having a proximal end and a distal end, said distal end being attached to the first compression bead component, and one or more lumens extending at least partially between said proximal and distal end; and
a source of electrical current sufficient to allow the electrically activated heating element to generate the temperature within the chamber greater than the melting temperature of the suture to be cut, wherein the source of electrical current is connected to the electrical leads and further wherein the source of electrical current includes a switch capable of interrupting a flow of electrical current, said source of electrical current being associated with the proximal end of the push rod;
wherein said first compression bead component and second compression bead component cooperate with the suture to be cut to form a substantially fluid tight chamber.

2. The thermal suture cutting device of claim 1, wherein the suture to be cut enters the substantially fluid tight chamber through the first opening defined by the first compression bead component, passes proximate to the electrically activated heating element within the substantially fluid tight chamber, and exits the substantially fluid tight chamber through the second opening defined by the second compression bead component.

3. The thermal suture cutting device of claim 1, further comprising a handle associated with the proximal end of the push rod, wherein the handle contains the source of electrical current.

4. The thermal suture cutting device of claim 1, wherein the first compression bead component is formed from a material not adversely affected by the temperature generated within the chamber by the electrically activated heating element.

5. The thermal suture cutting device of claim 4, wherein the material of the first compression bead component is a ceramic.

6. The thermal suture cutting device of claim 4, wherein the first compression bead component is formed from an electrical insulator.

7. The thermal suture cutting device of claim 1, wherein the second compression bead component is formed from a material not adversely affected by the temperature generated within the chamber by the electrically activated heating element.

8. The thermal suture cutting device of claim 7, wherein the material of the second compression bead component is a ceramic.

9. The thermal suture cutting device of claim 7, wherein the first compression bead component is formed from an electrical insulator.

10. A system for sealing a vascular puncture comprising:
an anchor deployable adjacent to a vascular puncture;
a suture attached to the anchor deployable adjacent to the vascular puncture;
a hemostatic material deployed about the suture; and
a thermal suture cutting device comprising:
a chamber further comprising:
a first compression bead component defining a first opening therethrough, said first opening being sized and adapted to slidingly receive a suture to be cut;
a second compression bead component defining a second opening therethrough, said second opening being sized and adapted to slidingly and sealingly receive the suture to be cut;
an electrically activated heating element within the chamber, said electrically activated heating element being capable of generating a temperature within the chamber greater than the melting temperature of the suture to be cut; and
electrical leads at least partially within the chamber capable of supplying sufficient current to the electrically activated heating element to allow the electrically activated heating element to generate a temperature within the chamber greater than the melting temperature of the suture to be cut;
a push rod having a proximal end and a distal end, said distal end being attached to the first compression bead component, and one or more lumens extending at least partially between said proximal and distal end; and
a source of electrical current sufficient to allow the electrically activated heating element to generate the temperature within the chamber greater than the melting temperature of the suture to be cut, wherein the source of electrical current is connected to the electrical leads and further wherein the source of electrical current includes a switch capable of interrupting a flow of electrical current, said source of electrical current being associated with the proximal end of the push rod;
wherein said first compression bead component and second compression bead component cooperate with the suture to be cut to form a substantially fluid tight chamber.

11. The system for sealing a vascular puncture of claim 10, wherein the anchor deployable adjacent to the vascular puncture is deployed adjacent to the vascular puncture and inside of a vessel defining the vascular puncture.

12. The system for sealing a vascular puncture of claim 10, wherein the suture to be cut enters the substantially fluid tight chamber through the first opening defined by the first compression bead component, passes proximate to the electrically activated heating element within the substantially fluid tight chamber, and exits the substantially fluid tight chamber through the second opening defined by the second compression bead component.

13. The system for sealing a vascular puncture of claim 10, further comprising a handle associated with the proximal end of the push rod, said handle containing the source of electrical current.

14. The system for sealing a vascular puncture of claim 10, wherein the first compression bead component is formed from a material not adversely affected by the temperature generated within the chamber by the electrically activated heating element.

15. The system for sealing a vascular puncture of claim 10, wherein the second compression bead component is formed from a material not adversely affected by the temperature generated within the chamber by the electrically activated heating element.

16. A method of cutting a suture in a fluid environment comprising:
passing a suture through a thermal suture cutting device comprising:
a chamber further comprising:
a first compression bead component defining a first opening therethrough, said first opening being sized and adapted to slidingly receive the suture;
a second compression bead component defining a second opening therethrough, said second opening being sized and adapted to slidingly and sealingly receive the suture;
an electrically activated heating element within the chamber, said electrically activated heating element being capable of generating a temperature within the chamber greater than the melting temperature of the suture; and
electrical leads at least partially within the chamber capable of supplying sufficient current to the electrically activated heating element to allow the electrically activated heating element to generate a temperature within the chamber greater than the melting temperature of the suture;
a push rod having a proximal end and a distal end, said distal end being attached to the first compression bead component, and one or more lumens extending at least partially between said proximal and distal end; and
a source of electrical current sufficient to allow the electrically activated heating element to generate the temperature within the chamber greater than the melting temperature of the suture, wherein the source of electrical current is connected to the electrical leads and further wherein the source of electrical current includes a switch capable of interrupting a flow of electrical current, said source of electrical current being associated with the proximal end of the push rod;
wherein said first compression bead component and second compression bead component cooperate with the suture to be cut to form a substantially fluid tight chamber;
positioning the suture and the thermal suture cutting device in a fluid environment;
and passing a current through the electrically activated heating element sufficient to allow the electrically activated heating element to generate a temperature within the chamber greater than the melting temperature of the suture.

17. The method of cutting a suture in a fluid environment of claim 16, further comprising removing the thermal suture cutting device from the fluid environment.

18. The method of cutting a suture in a fluid environment of claim 16, wherein the fluid environment, wherein the fluid comprises a body fluid.

19. The method of cutting a suture in a fluid environment of claim 18, wherein the body fluid comprises blood.

20. The method of cutting a suture in a fluid environment of claim 16, wherein the step of passing a current through the electrically activated heating element includes closing a switch.
